(19) United States
(12) Patent Application Publication
(10) Pub. No.: US 2013/0125902 A1
(43) Pub. Date: May 23, 2013

(54) SLEEP APNEA RELIEF SYSTEM

(71) Applicant: Armen Vahe Danielian, Woodland Hills, CA (US)

(72) Inventor: Armen Vahe Danielian, Woodland Hills, CA (US)

(21) Appl. No.: 13/679,150

(22) Filed: Nov. 16, 2012

Related U.S. Application Data
(60) Provisional application No. 61/561,250, filed on Nov. 17, 2011.

(51) Int. Cl. A61B 17/24

(52) U.S. Cl. CPC A61B 17/24
USPC 128/859

(57) ABSTRACT

Described is an oral appliance for providing sleep apnea relief. The appliance includes a body formed in a u-shape ending in a rear portion having a first end and a second end. A malleable metal spine is molded into the body and protrudes from at least one of the first and second ends. A tongue depressor is connected with the malleable spine, thereby allowing a user to bend the malleable spine to position the tongue depressor into the desired position.

### CROSS-REFERENCE TO RELATED APPLICATIONS

This is a Non-Provisional Utility patent application of U.S. Provisional Application No. 61/561,250, filed on Nov. 17, 2011, entitled, “Sleep Apnea. Relief System.”

### BACKGROUND OF THE INVENTION

(1) Field of Invention

The present invention relates to a mouth piece and, more particularly, an oral appliance that is specifically designed to relieve the symptoms of sleep apnea.

(2) Description of Related Art

Sleep Apnea is a sleep disorder characterized by abnormal pauses in breathing during sleep. Each pause in breathing, called an apnea, can last from a few seconds to minutes and can occur several times an hour. There are a few forms of sleep apnea. One commonly understood form is obstructive sleep apnea (OSA). In OSA, breathing is interrupted by a physical block to airflow despite respiratory effort. Snoring is also a common effect of OSA. As one can image, abnormal pauses during breathing can result in several symptoms, such as excessive daytime sleepiness (EDS) and impaired alertness.

There are a few existing oral appliances for snoring and apnea relief, but they all require custom fabrication and lengthy fittings by the doctors and dental lab workers and typically require multiple visits to the doctors' offices. Due to their required customization, the existing devices are very expensive and not suitable for the masses that may suffer from apnea.

Additionally, a few of the products in this category use a bridge across the end of the teeth arch. This bridge creates a dam and blocks the flow of saliva to the esophagus. This saliva damming creates a gargling and or gagging effect on the patient.

Thus, a continuing need exists for an oral appliance that provides relief from snoring and/or apnea. Specifically, a need exists for such a device that addresses the above mentioned issues by eliminating the dental lab work and reducing the number of fitting iterations significantly by using a mass produced (not custom fitted) appliance that is fully adjustable and can be quickly fitted to the patient's mouth and tongue. Further, a continuing need exists for an oral appliance that funnels the saliva away to eliminate the gargling and gagging effect.

### SUMMARY OF INVENTION

The present invention relates to an oral appliance for providing sleep apnea relief. In one aspect, the oral appliance includes a body formed in a u-shape ending in a rear portion having a first end and a second end. The body includes a base layer sandwiched between a top layer and a bottom layer, wherein the top and bottom layers are formed of a heat formable plastic that is adapted to molded upon exposure to boiling water. A first cross bracket connects the first and second ends, with a second cross bracket also connecting the first and second ends. The first and second cross brackets are adapted to operate as structural elements to support a tongue depressor. Additionally, a tongue depressor is included for connecting with at least one of the first and second cross brackets.

The body includes two base connectors and the tongue depressor includes two tongue connectors for selectively connecting with the corresponding base connector. The tongue depressor also includes a tongue catch for connecting, with the first cross bracket. Further, the tongue depressor includes a rear tongue connector for connecting with the second cross bracket. Thus, when the tongue depressor connects with the body, it is adapted to be snapped into mating engagement with the body and pivoted about a snap axis.

In another aspect, a malleable spine (e.g., metal) is molded into the body and protrudes from at least one of the first and second ends. In this aspect, a tongue depressor is connected with the malleable spine, thereby allowing a user to bend the malleable spine to position the tongue depressor into the desired position.

In yet another aspect, the tongue depressor has a hole formed therethrough to allow saliva to pass through the tongue depressor.

In another aspect, the spine includes a retention portion and the tongue depressor is rotatably connected with the retention portion. Additionally, the spine includes a plurality of protrusions of differing lengths, whereby a user can selectively adjust the tongue depressor by rotating the tongue depressor until the desired length is selected at a position to engage with a tongue when in use. Further, the spine is connected to and between the first and second ends of the rear portion.

In yet another aspect, the spine protrudes separately from each of the first and second ends of the rear portion such that the spine includes two separate retention portions, each with its own tongue depressor connected thereto. In this aspect, each of the tongue depressors is adapted to be bent and extended independently to push on a user's tongue at different points.

in another aspect, the tongue depressor is formed to include a funneling trough to allow for the drainage of saliva.

In yet another aspect, the spine is spring biased to three the tongue depressor down and against a user's tongue. Additionally, an articulation point, exists between the tongue depressor and body, such that both a cross-section of the body and tongue depressor at the articulation point is circular to provide a smooth exterior surface at all angular settings.

In another aspect, a tongue depressor is rotatably connected with the body proximate each of the first and second ends. As was the case above, an articulation point exists between the tongue depressor and body, such that both a cross-section of the body and tongue depressor at the articulation point is circular to provide a smooth exterior surface at all angular settings.

In another aspect, each of the first and second ends of the body include a connector. In this aspect, the tongue depressor includes connectors for selectively connecting with the connectors on the first and second ends, whereby a user can selectively select a tongue depressor with the desired dimensions and attached the selected tongue depressor with the body.

Finally, as can be appreciated by one in the art, the present invention also comprises a method for forming and using the invention described herein.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1A is a perspective-view illustration of a fully assembled oral appliance according to the present invention;

FIG. 1B is cross-sectional view of the oral-appliance depicted in FIG. 1A, taken from line A-A;

FIG. 1C is a perspective-view illustration of a structural metal spine as used in the oral appliance depicted in FIG. 1A;

FIG. 2A is a perspective-view illustration of a fully assembled oral appliance according to the present invention;

FIG. 2B is a perspective-view illustration of a structural metal spine as used in the oral appliance depicted in FIG. 2A;

FIG. 2C is a side-view illustration of the oral appliance;

FIG. 3A is a perspective-view illustration of a hilly assembled oral appliance according to the present invention;

FIG. 3B is a perspective-view illustration of a structural metal spine as used in the oral appliance depicted in FIG. 3A;

FIG. 4 is a perspective-view illustration of an oral appliance according to the present invention;

FIG. 5 is a perspective-view illustration of an oral appliance according to the present invention;

FIG. 6A is a perspective-view illustration of an oral appliance according to the present invention;

FIG. 6B is a top-view illustration of the oral appliance depicted in FIG. 6A;

FIG. 6C is a side-view illustration of the oral appliance depicted in FIG. 6B, shown from line B-B;

FIG. 7A is a perspective-view illustration of an oral appliance according to the present invention;

FIG. 7B is a top-view illustration of the oral appliance depicted in FIG. 7A;

FIG. 7C is a cross-sectional view of the oral appliance depicted in FIG. 7B, shown from line C-C;

FIG. 8A is a perspective-view illustration of an oral appliance according to the present invention;

FIG. 8B is a top-view illustration of the oral appliance depicted in FIG. 8A;

FIG. 8C is cross-sectional view of the oral-appliance depicted in FIG. 8B, taken from line D-D; and

FIG. 8D is a cross-sectional view of the oral appliance, showing a tongue depressor affixed with the body and first and second bridges.

FIG. 8E is a front-view illustration of the oral appliance depicted in FIG. 8A;

FIG. 8F is a top-view illustration of the oral appliance depicted in FIG. 8A;

FIG. 8G is a side-view illustration of the oral appliance depicted in FIG. 8A;

FIG. 8H is a cross-sectional view of the oral appliance, showing the tongue depressor being progressively replaced with longer tongue depressors at increasing angles; and

FIG. 9 is a top-view illustration of an oral appliance according to the present invention.

### DETAILED DESCRIPTION

The present invention relates to a mouth piece and, more particularly, an oral appliance that is specifically designed to relieve the symptoms of sleep apnea. The following description is presented to enable one of ordinary skill in the art to make and use the invention and to incorporate it in the context of particular applications. Various modifications, as well as a variety of uses in different applications will be readily apparent to those skilled in the art and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being, limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is only one example of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

Please note, if used, the labels left, right, front, back, top, bottom, forward, reverse, clockwise and counter clockwise have been used for convenience purposes only and are not intended to imply any particular fixed direction. Instead, they are used to reflect relative locations and/or directions between various portions of an object.

### (1) INTRODUCTION

As noted above, the present invention is directed to an oral appliance that is specifically designed to relieve the symptoms of sleep apnea. Described herein are several aspects of the oral appliance, all of which are a substantive improvement over the prior art. The aspects are similar in that they can be manufactured and quickly fitted to the patient teeth. This is the first dental oral appliance with a tongue depressor that can be quickly adjusted to the patient's teeth and tongue position and orientation for use by patients who suffer from snoring and/or obstructive sleep apnea. The tongue depressor on this appliance keeps the airway open to allow the patient to breathe freely and minimize or eliminate sleep apnea, in addition to minimizing or eliminating snoring. While described herein are several different aspects of the oral appliance, they are similar in that they each cover the lower teeth and the upper molars (but is not anchored to them) and have a quickly adjustable tongue depressor that keeps the tongue down to maximize airflow to the lungs.

For further understanding, specific aspects of the present invention are described in more detail below.

### (2) SPECIFIC ASPECTS

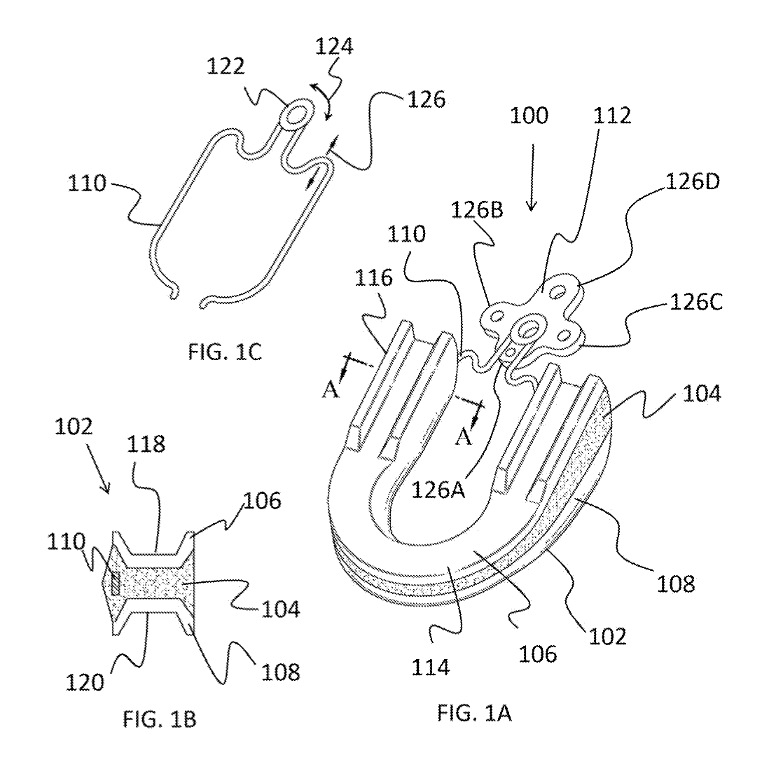

Illustrated in FIGS. 1A through 1C is a first aspect of an oral appliance 100 of the present invention. In this aspect and as shown in FIG. 1A, the oral appliance 100 includes a body 102 that is formed to fit over or otherwise engage with the user's teeth. The body 102 can be formed of a single material or multiple layers of different materials. As a non-limiting example, the body 102 can be formed to include a heat stable base layer 104 that, although suitably pliable, generally maintains its form when exposed to heat (such as boiling water). An example of such a material is an acrylic plastic. Alternatively, sandwiching the base layer 104 is a top layer 106 and a bottom layer 108 (that can be separately formed and attached or molded onto the base layer 104). The top 106 and bottom 108 layers can be formed of a heat formable plastic that allows for customization of the oral appliance 100. For example, the top 106 and bottom 108 layers are formed of a plastic material that allows for the layers 106 and 108 to become soft and pliable when exposed to hot water and, once removed and cooled, assume their new form. A specific non-limiting example of such a material is Elvax, as produced by Dupont, located at 1007 Market Street, Wilmington, Del. 19898.

Additionally, a spine (e.g., metal spine) 110 is inserted (e.g., molded into) the base layer 104. Finally, a tongue depressor 112 is attached with the spine 110. The tongue depressor 112 is formed of any suitably non-toxic and stable material, a non-limiting example of which includes plastic. Further, the spine 110 is formed of any suitable malleable material that allows the spine 110 to be adjusted to provide for adjustment of the actual location of the tongue depressor 112. As a non-limiting example, the spine 110 is formed of a malleable stainless steel metal that provides the structural integrity to the appliance 100 and provides the quick angular adjustability feature to the tongue depressor 112. The structural integrity of this metal spine 110 provides the tension that pushes down on the tongue with the tongue depressor 112.

For further understanding. FIG. 1B provides a cross-sectional view of the body 102, illustrating the base layer 104 sandwiched between the top 106 and bottom 108 layers. Also shown is the spine 110 that is insert-molded into the base layer 104 (i.e., the base layer 104 is molded around the spine 110). Referring again to FIG. 1A, it should be noted that the body 102 includes a front portion 114 and a rear portion 116, in which the rear portion 116 is optionally formed to mold around the user's molars. For example and as shown in FIG. 18 (which is a cross-sectional view of the rear portion), the oral appliance can be formed such that it includes a top channel 118 and a bottom channel 120, which are formed to accommodate the top and bottom molars, respectively. While the front portion can be similarly formed to include channels, it is optional and not illustrated in the figures.

FIG. 1C provides an illustration of the spine 110. As noted above, the spine 110 is adjustable to allow for adjustment of the tongue depressor (shown as element 112 in FIG. 1A). The spine 110 includes a retention portion 122, around or within which is attached the tongue depressor. Due to the malleability of the spine, the retention portion 122 (and the attached tongue depressor) can be adjusted by bending the spine 110 to move the retention portion 122 angularly up-or-down 124 or forward-or-backwards 126.

Further and referring again to FIG. 1A, the tongue depressor 112 can be formed to allow for rotational adjustment. In this aspect, the tongue depressor 112 is detented at every 90 degrees of rotation with protrusions (i.e. 126A, 126B, 126C, and 126D). In other words, in this aspect, the tongue depressor 112 is centrally and rotatably attached with the retention portion 122, around which the tongue depressor 112 can rotate. The tongue depressor 112 is rotatably attached with the retention portion 122 using any suitable mechanism or technique, a non-limiting example of which includes forming the tongue depressor 122 to have a central mushroom-shaped plug that can be pressed into and through the retention portion 122. Because each of the protrusions (i.e., 126A, 126B, 126C, and 126D) are in increasing lengths, every 90 degrees of rotation provides an increasing amount of protrusion into the hack of the tongue to maximize the airflow to the lungs.

In another aspect, the tongue depressor 112 is formed with holes therethrough that allow for saliva, to flow through and around the tongue depressor 112 to prevent accumulation of fluids and therefore eliminate gagging. Thus, because the tongue depressor 112 has an open palate design (due to its shape and holes), the oral device 100 provides a funneling passageway for the saliva to flow through due to gravity to the esophagus, thereby eliminating the gargling and gagging effect. In addition to draining the saliva this trough increases the airway cross-section to the lunges.

As can be appreciated by one skilled in the art, due to the ability to quickly customize the position of the tongue depressor 112, the oral appliance 112 of the present invention is a considerable improvement over the prior art. First, it is quickly fitted to the patient's teeth using, a plastic (on the top 106 and bottom 108 layers) that would soften when heated in hot water. The areas (i.e. the top 106 and bottom 108 layers) that would contact the user's teeth are made out of a hot water softened plastic. To form to the user's teeth, the oral appliance 100 would be placed in hot water which would soften this plastic. Then the appliance is placed in cold water for a short period of time to cool the exposed surfaces to eliminate flesh burns that may be caused by the hot pliable plastic. This surface cooling would not affect the form-ability of the plastic. Once the exposed surfaces are cooled to some extent, the appliance is placed in the patent's mouth and fitted to the patent's upper and lower teeth at the same time.

Next, the tongue depressor 112 is adjusted to fit the position and angle of the patent's tongue and provide tension to push the tongue down, at the optimum location, to keep the airway open. This tongue depressor's protrusion (i.e., 126A, 126B, 126C, or 126D) into the back of the tongue can be adjusted by rotating it. Every 90 degrees of rotation provides an increasing amount of protrusion into the back of the tongue to maximize the airflow to the lungs.

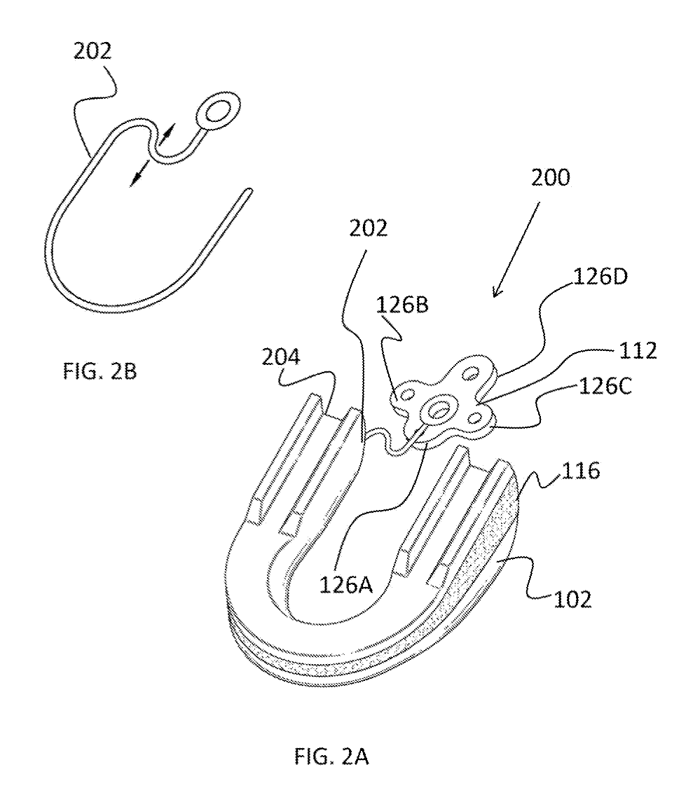

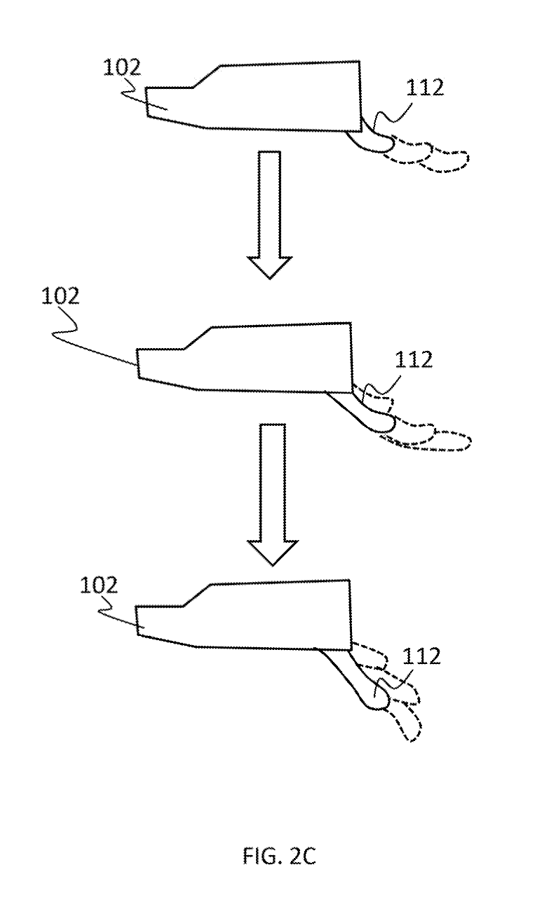

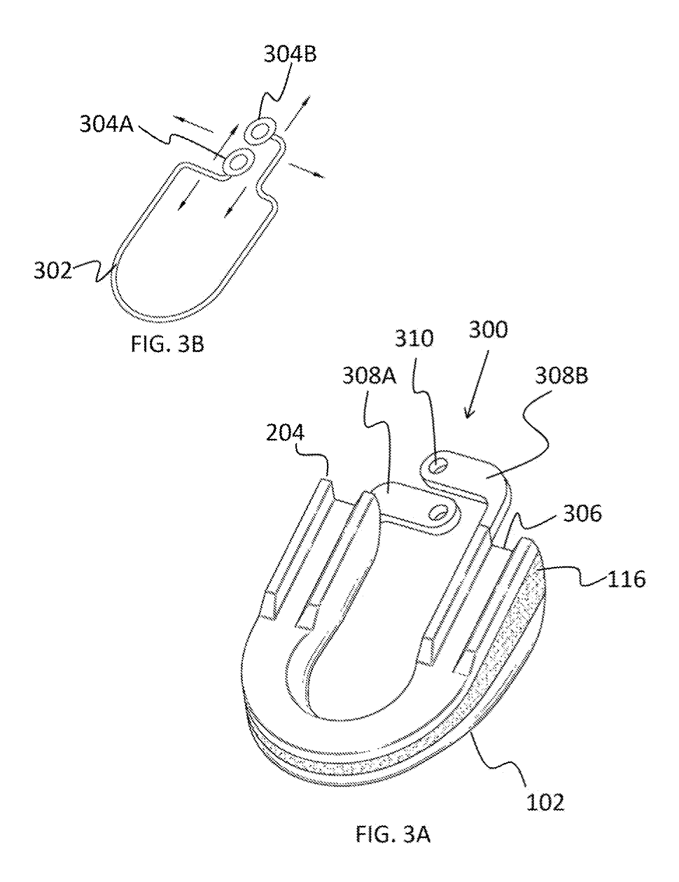

Illustrated in FIGS. 2A through 3B are additional aspects of the oral appliance. Although they are similar in many ways as the design illustrated in FIG. 1A, they differ in the shaping of the metal spine and tongue depressor and the manner by which the tongue depressor can be adjusted.

Specifically, FIGS. 2A and 2B illustrate another aspect of an oral appliance 200. In this aspect, the spine 202 is similarly molded into the body 102. However, to be contrasted with the aspect illustrated in FIG. 1A, the spine 202 in this aspect extends from the body 102 from only a first end 204 of the rear portion 116 (whereas in the aspect of FIG. 1A, the spine 110 bridged both ends of the rear portion 116). As was the case above and as illustrated in the side-view illustration of FIG. 2C, the tongue depressor 112 includes protrusions (i.e., 126A, 126B, 126C, or 126D) of different lengths that allow for rotational adjustment of the tongue depressor 112 and, thereby, increasing amount of protrusion (with respect to the body 102) down and back toward the back of the tongue to maximize the airflow to the lungs.

Another aspect of the oral appliance 300 is illustrated in FIGS. 3A and 3B. In this aspect, the spine 302 includes two separate retention portions 304A and 304B that are joined via a u-shaped frame. The spine 302 is insert molded into the base 102 such that the retention portions (elements 304A and 304B are not depicted in FIG. 3A) protrude from each of the first end 204 and second end 306, respectively, of the rear portion 116. It is worth noting that the body 102 described throughout this description is generally formed in a u-shape (or horse-shoe shape) ending in a rear portion 116 having the first end 204 and a second end 306. The distinction between the various aspects described, herein largely rests on the tongue depressor and how it is connected with one of or both of the first 204 and second 306 ends.

In this aspect, molded onto each of the retention portions is a tongue depressor 308A and 308B. Although the tongue depressors 308A and 308B can be formed in any suitable shape, they are desirably formed into a paddle-shape, as illustrated. The tongue depressors 308A and 308B can be bent and extended independently to push on the tongue at different points to optimize the appliance's effectiveness. As was the case above, each of the tongue depressors 308A and 308B also can be formed with a hole 310 therethrough that allows for saliva to flow through and around to prevent accumulation of fluids and eliminate gagging.

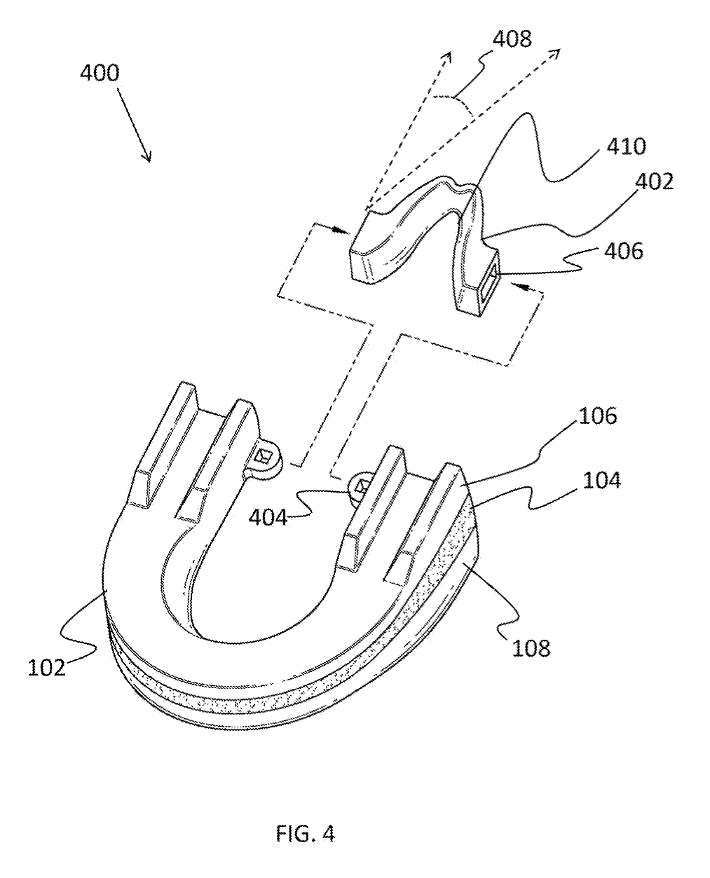

Another aspect of the oral appliance 400 is illustrated in FIG. 4. As was the case above, in this aspect, the body 102 also includes a base layer 104 sandwiched between the top 106 and bottom 108 layers. However, in this aspect, the tongue depressor 402 is separately provided, and can thereafter be attached to the body 102 using any suitable attachment technique. As a non-limiting example, the body 102 includes a male connector 404 that can snap connect into a corresponding female connector 406 (on the tongue depressor 402).

The body 102 can be provided in many sizes to fit a particular patient's teeth and arch size. Further, the tongue depressor 402 in this aspect can also be provided in many sizes, including multiple lengths and angular orientations. In operation, a doctor could first size the patent's teeth arch size. Then he would choose a body 102 that would fit the patient best. Thereafter, the doctor would choose a desired tongue depressor 402. As a non-limiting example, the doctor could select the shortest tongue depressor 402 available with the least steep angle 408, which could then be snap connected to the body 102. In other words, the tongue depressors 402 can be provided in varying lengths and degrees to accommodate different tongue configurations.

Then the appliance can be fitted to the patient's teeth as follows. As was the case above, the body 102 is fitted to the lower and upper teeth of the patient using a plastic that would soften up when heated in hot water. The areas that would contact the user's teeth are molded out of this hot water softened plastic. To mold to the user's teeth, the oral appliance 400 would be placed in hot water which would soften the plastic. Then the appliance is placed in cold water for a short period of time to cool the exposed surfaces to eliminate flesh burns that may be caused by the hot pliable plastic. This surface cooling, would not affect the mold-ability of the plastic. Once the exposed surfaces are cooled to some extent, the appliance is placed in the patent's mouth and fitted to the patent's teeth and allowed to harden into a stable form.

Thereafter, the doctor could unsnap the tongue depressor 402 and snap another tongue depressor with the proper angle and length onto the male connector 404. This last step of selectively changing the tongue depressor 402 may have to be repeated a few times to optimize the effectiveness of the appliance by selecting the tongue depressor 402 with the desired length and angle properties to depress the tongue. It should be noted that the tongue depressor 402 includes a funneling passageway 410 that allows for drainage of saliva.

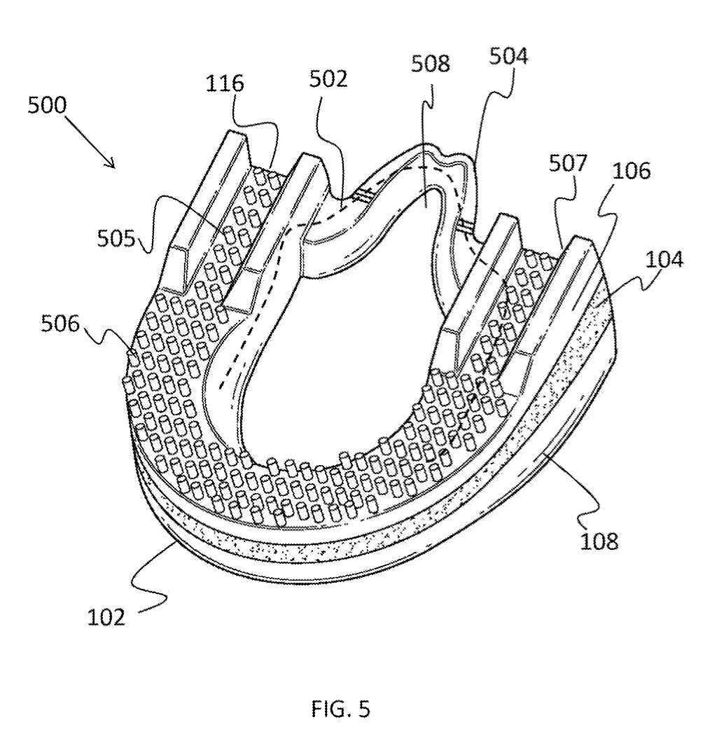

Another aspect of an oral appliance 500 is depicted in FIG. 5. As was the case above, in this aspect, the body 102 also includes a base layer 104 sandwiched between the top 106 and bottom 108 layers. The design depicted in FIG. 5 represents an over the counter snoring relief appliance that could be very easily adjusted by the end user to his teeth.

In this aspect, a malleable metal spine 502 is molded into the base and spans the rear portion 116 of the body 102. A tongue depressor 504 is molded around the spine 502. Pliable members 506 can also be formed on the exterior surfaces of each of the top 106 and/or bottom 108 layers. The pliable members 506 are any suitable protrusion that is pliable and formed to accommodate teeth of various sizes and shapes, a non-limiting example of which includes a plurality of columnar protrusions or nubs. In one aspect, the oral appliance 500 can be formed of a plastic that allows for heat casting (as was the case above). However, in another aspect, the body 102 (with the pliable members 506) can adapt and fit the user's teeth without the use of heat or casting materials because at least one of the top 106 and/or bottom 108 layers with the teeth profile channels 507 (in conjunction with the pliable members 506) are engineered to be flexible enough to allow them to adjust to the users teeth.

Further, the malleable metal spine 502 that is molded into the plastic allows the position of the molar portion 505 (i.e., the portion of the body 102 that engages the molars) and the angle of the tongue depressor 504 to be adjusted by the customer. As was the case above, the oral appliance 500 includes a funneling trough 508 to move the saliva from the front of the mouth to the esophagus.

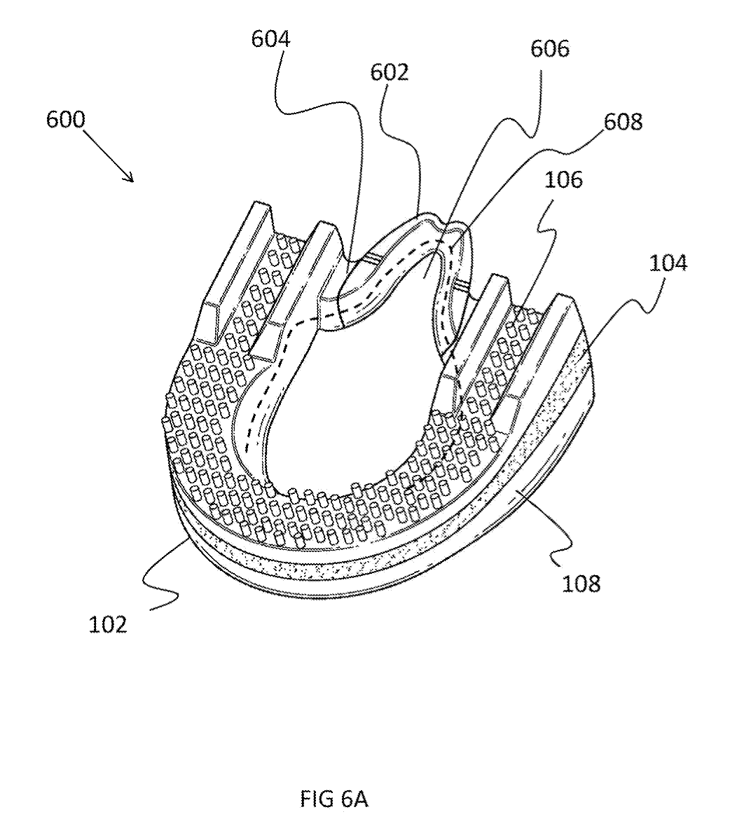

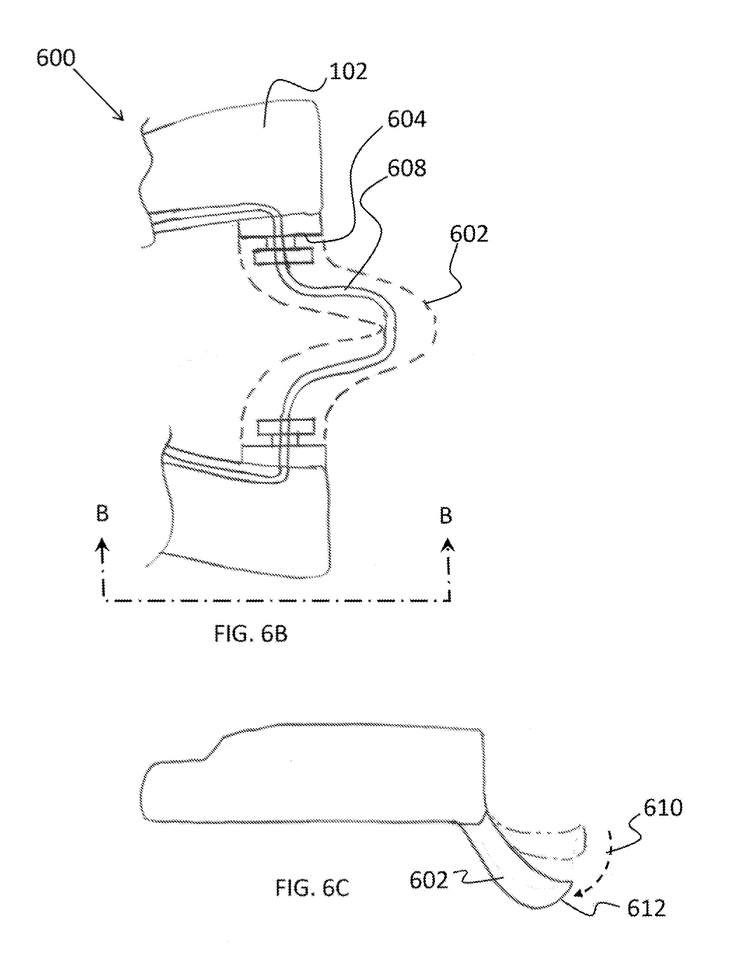

FIG. 6A illustrates another aspect of the oral appliance 600. As was the case above, in this aspect, the body 102 also includes a base layer 104 sandwiched between the top 106 and bottom 108 layers. Although the aspect depicted in FIG. 6A is nearly identical to that illustrated in FIG. 5, it differs in that the tongue depressor 602 is articulated (at an articulation point 604) and spring loaded. For example, the body 102 can be formed first, with the tongue depressor 602 insert molded around a biased spine 608 to form the articulation point 604 therebetween. Further, it uses the same funneling trough 606 as described above. This aspect is further illustrated in FIG. 6B, which is a top-view illustration of the oral appliance 600, illustrating an interior of the tongue depressor 602 and the biased spine 608. As shown, the articulation point 604 is formed between the body 102 and tongue depressor 602.

As shown in the side-view illustration of FIG. 6C, the spring-loaded tongue depressor 602 adjusts to the proper tongue angle automatically by being biased 610 (via the tension or biasing properties of the biased spine) toward the tongue when in use. In other words, when in use, the tongue will naturally push the tongue depressor 602 up; however, because of the biased spine 608, the tongue depressor 602 has a tendency to push the tongue toward its original position 612 (via the biased spine 608).

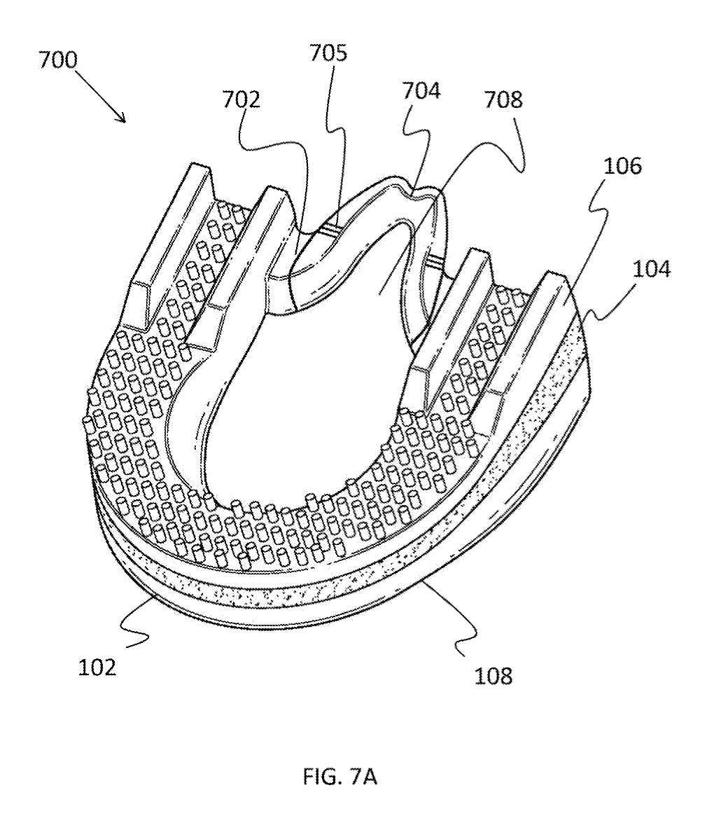

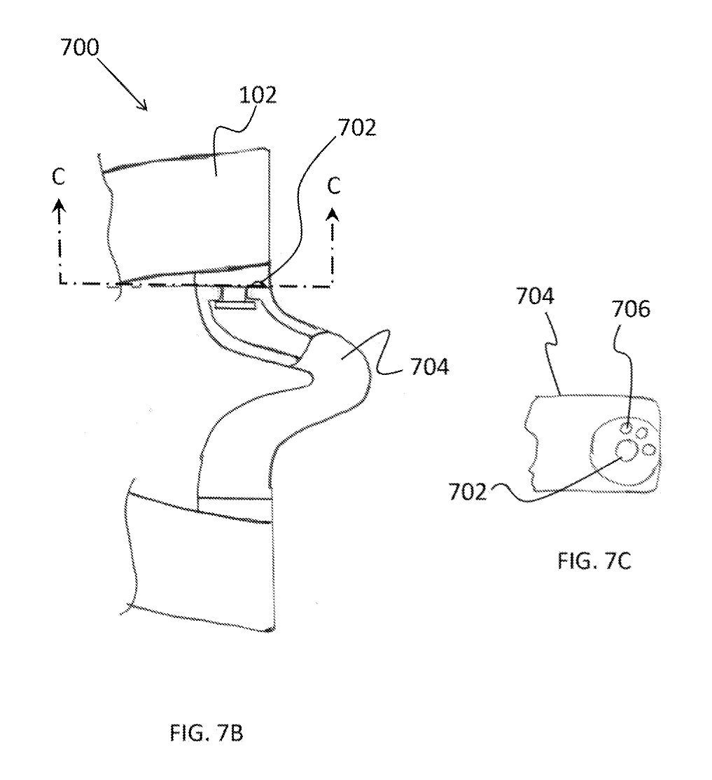

Another aspect of an oral appliance 700 is depicted in FIG. 7A. As was the case above, in this aspect, the body 102 also includes a base layer 104 sandwiched between the top 106 and bottom 108 layers. Although the aspect depicted in FIG. 7A is nearly identical to that as illustrated in FIG. 6A, it differs in that it has an articulated (at an articulation point 702) and adjustable tongue depressor 704 with multiple preset detented positions (which can be monitored via detention markings 705). While it can be optionally formed with a spine molded within the tongue depressor 704, it is desirably formed without the spine and, instead, includes detentions to allow for selective adjustment.

As shown in the top-view illustration of FIG. 7B, the body 102 is attached with the tongue depressor 704 via the articulation point 702, which operates as a hinge to allow for selective adjustment (i.e., rotation) of the tongue depressor 704. As shown in the cross-sectional view of FIG. 7C, at the articulation point 702, there can be detentions 706 to allow adjustment of the angle of the tongue depressor 704. A non-limiting example of the detentions include dimples in the body 102 and corresponding nubs on the tongue depressor 704 to fix the position of the tongue depressor 704 as it is rotated about the articulation point 702. The tongue depressor 704 can be molded out of a different material than the body 102 with a lower melting point, than the body 102 (or vice versa).

The patient adjusts this tongue depressor 704, to a specific position, to optimize the appliance 700 to the user's tongue and comfort level. Further, it uses the same funneling trough 708 as described above.

It should be noted that the articulation point in the aspects depicted in both FIGS. 6B and 7B can be formed in an suitable shape to accommodate a suitable rotation between the body and tongue depressor. As a non-limiting example, both the cross-section on the body and the tongue depressor are circular to provide a smooth exterior surface at all angular settings. This surface is desirably circular only exactly at the separation point or articulation point.

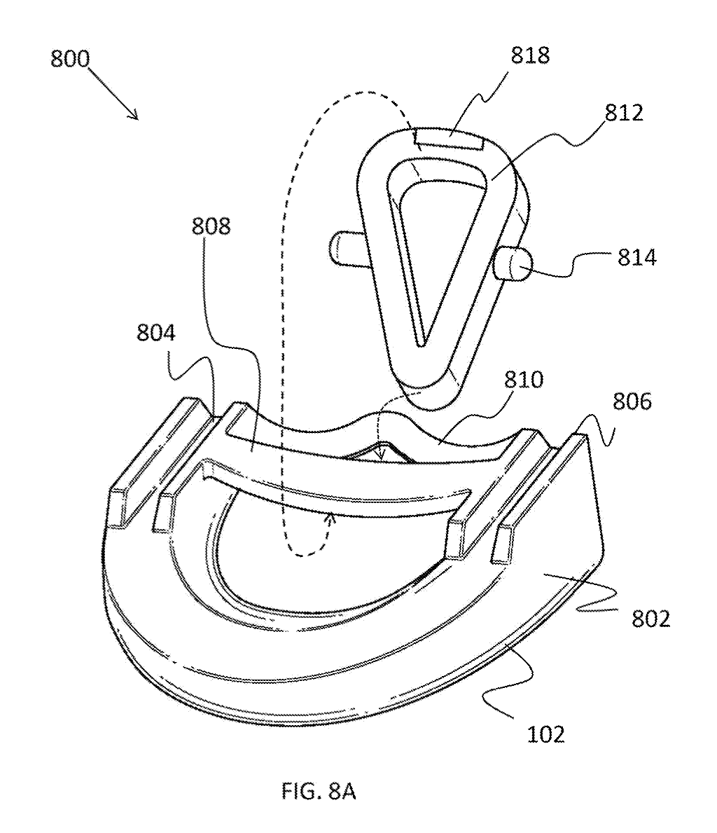

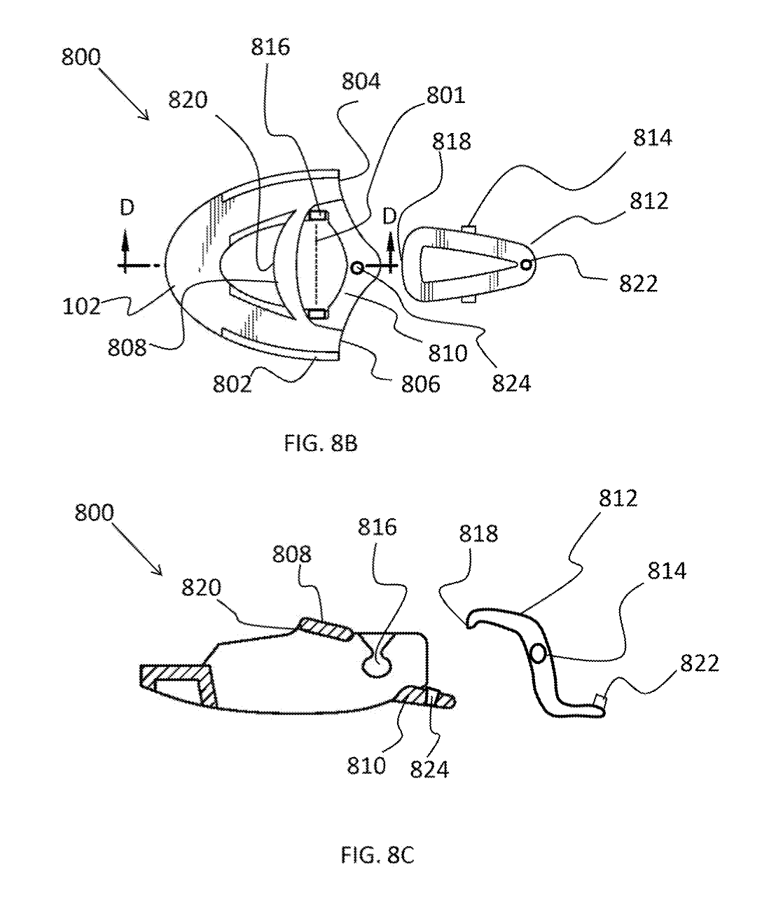

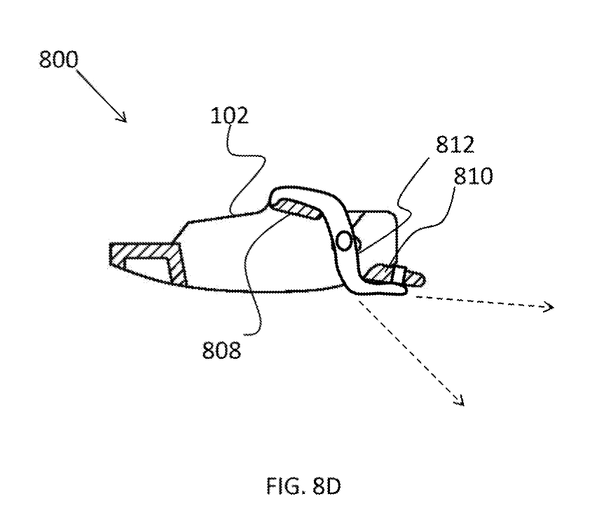

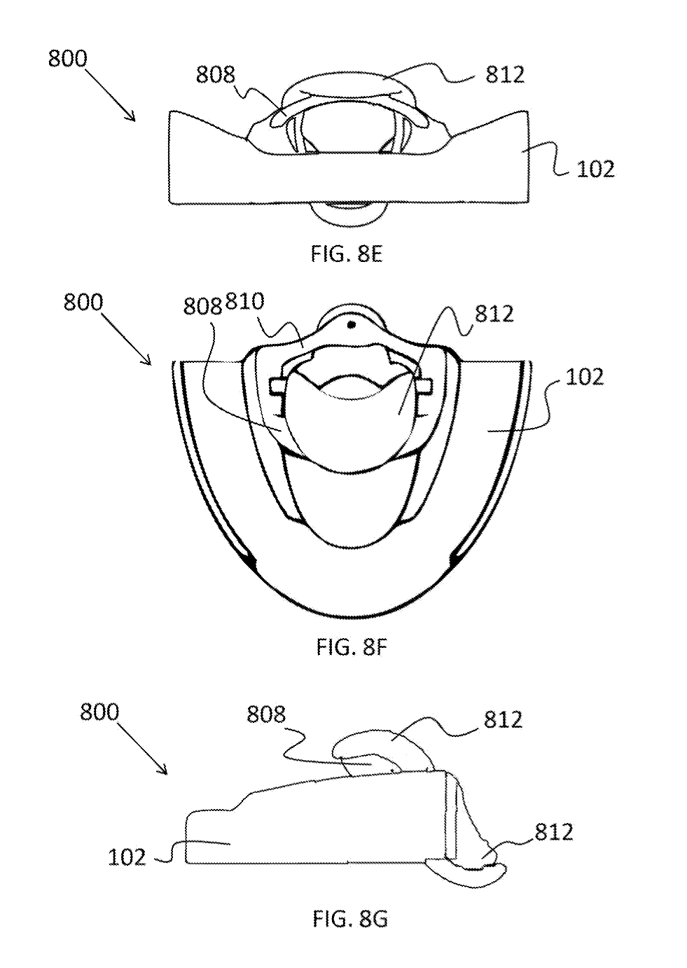

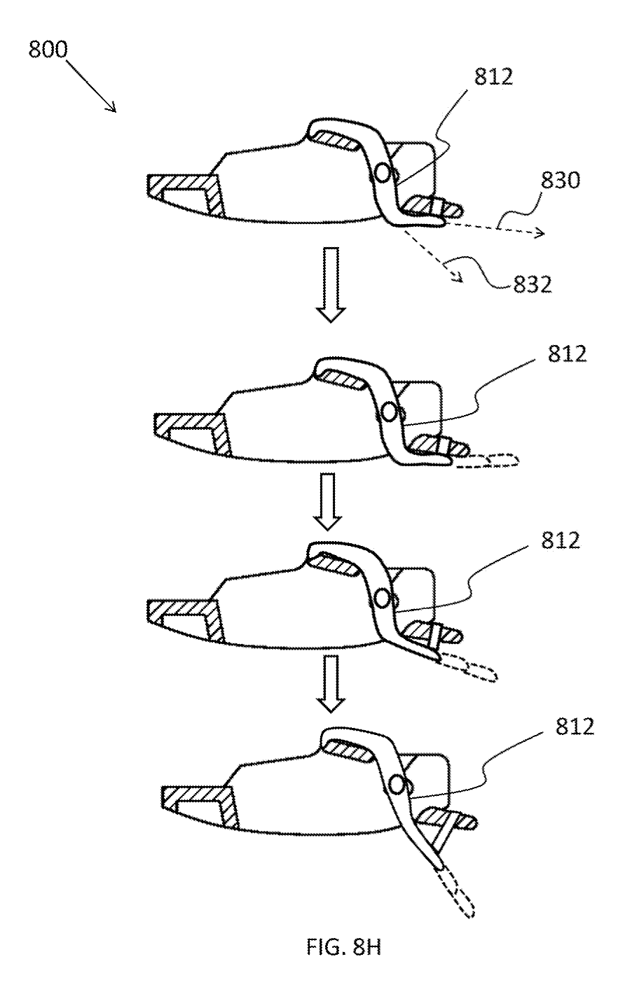

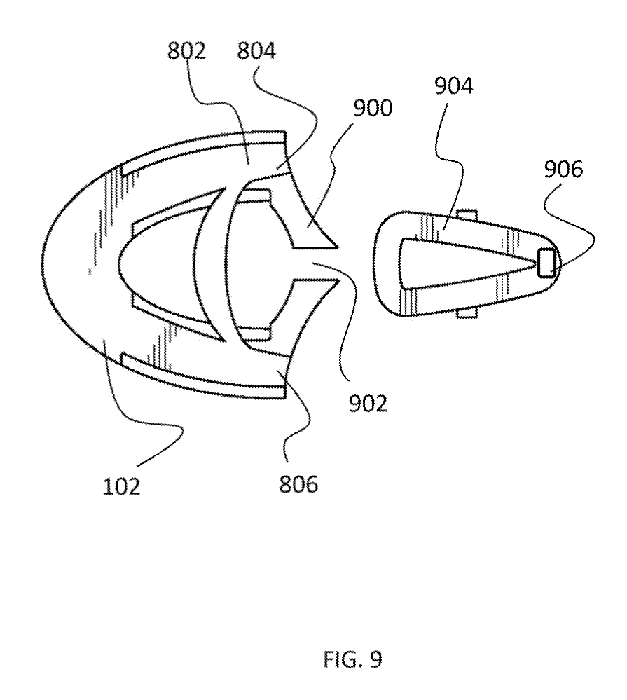

FIGS. 8A through 8H illustrate yet another aspect of an oral appliance 800 according to the present invention. In this aspect, the body 102 is formed in a horseshoe-shape that ends at a rear portion 802 having a first end 804 and second end 806. The body 102 can be formed of a single material or have multiple layers formed of different materials (such as to base layer sandwiched between top and bottom layers, as described above). Although not illustrated in FIG. 8A as being formed of multiple layers, it should be understood that the body 102 is formed of the same layers (material, etc.) as depicted and described above with respect to FIGS. 1A and 1B (i.e., the oral appliance as shown in FIG. 8A includes the same base layer 104 sandwiched between the top 106 and bottom layers 108, with the top 106 and bottom 108 layers formed of a heat formable plastic that allows for customization of the oral appliance 100).

Connecting the rear portion 802 across the first 804 and second ends 806 is a first cross bracket 808 and second cross bracket 810. The first 808 and second 810 cross brackets are transpalatal members that operate as structural elements to support a desired tongue depressor 812. The invention allows for the manufacture of multiple tongue depressors 812, each having a different length and angle setting. This will, allow the end user to select the desired tongue depressor 812 for attaching with the cross brackets 808 and 812 to optimize the air passage to lungs.

The tongue depressor 812 is designed to be held securely on the body 102 by securing the tongue depressor 812 against at least one of the cross brackets 808 or 810 using any suitable connection technique. As a non-limiting example, three points of securing engagement are provided between the tongue depressor 812 and the body 102 and cross brackets 808 and 810. For example, these points of engagement are as follows in the order they occur. First, a tongue connector 814 is connected with a base connector 816. The tongue connectors 814 and base connectors 816 are any suitable mechanism or device that allows for selective connection of two components. As a non-limiting example, the tongue connectors 814 are protrusions that protrude from the side of the tongue depressor 812 while the base connectors 816 are slots formed in the body 102 for receiving the protrusions in a snap-fit manner. Thus, the tongue connectors 814 are snapped into the base connectors 816 in a mating engagement.

Thereafter, the tongue depressor 812 can be rotated forwardly (around a snap axis 801) to snap a tongue catch 818 into engaging contact with a cross bracket catch 820 (on the first cross bracket 808). The tongue depressor 812 can be further secured by connecting a rear tongue connector 822 with a rear cross bracket connector 824. As is the case throughout this description, the actual connection techniques can be modified and still be within the scope of the present invention. Thus, as a non-limiting example, the rear tongue connector 822 is a protrusion that can be snap-fit into a hole that operates as the rear cross bracket connector 824.

By connected the tongue depressor 812 between the first 808 and second 810 cross brackets, the particular tongue engagement angle and length is set. Desirably, the end user would start by selecting the least steep and the shortest tongue depressor. Then gradually, after a few days or weeks, the user would change the tongue depressor 812 to a longer one and/or steeper one until the snoring or the apnea symptoms stop. It should be understood that the optimum engagement will be at different lengths and angles for different people. As such, the present invention allows for easy customization to achieve the desired result.

For further understanding, FIG. 8D illustrates a cross-sectional view of the oral appliance, showing the tongue depressor 812 affixed with the body 102 and first and second cross brackets 808 and 810. Additional images of the tongue depressor 812 affixed with the body 102 and first and second cross brackets 808 and 810 are provided in FIGS. 8E, 8F, and 8G, which provide a front-view, top-view, and side-view illustrations, respectively, of the oral appliance 800.

As noted above, it is desirable to allow a user to selectively modify the oral appliance to increase the length and angle at which the tongue depressor 812 engages with the user's tongue. For example and as shown in FIG. 8H, the user can selectively replace the tongue depressor 812 with progressively longer 830 tongue depressors 812 and at increasing angles 832. Thus, a kit can be sold that includes an oral appliance 800 with a collection of tongue depressors 812, each with a different length and angle to allow the user to progressively depress the tongue.

It should be noted that although the oral appliance as depicted in FIG. 8A is described as having a second cross bracket 810 connecting the rear portion 802 across the first 804 and second ends 806, the invention is not intended to be limited thereto, as depicted in the aspect illustrated in FIG. 9. As shown in FIG. 9, the second cross bracket can be replaced with a broken bracket 900 with a gap 902 formed through the broken bracket 900. In this aspect, the broken bracket 900 does not connect the rear portion 802 across the first 804 and second ends 806. When the tongue depressor 904 in this aspect is connected with the body 102, it can be pivoted to rest against the broken bracket 900 or, optionally, can include a connector (e.g., plug 906) that fills and matingly engages with the gap 902.

Finally and as noted above, the treatment of the apnea condition is accomplished by pressing down on the tongue, and preventing it from closing the airway to the lungs while someone is asleep. The reason there are different length and angled tongue depressors is to acclimate the patient gradually from the shortest and least steep tongue depressor to the longest and most steep one. Otherwise, the patient could possibly gag and would not be able to keep the appliance in his/her mouth while asleep. Multiple connection points (e.g., snaps) are provided to eliminate the risk of the tongue depressor coming loose and causing a choking hazard.

What is claimed is:

1. An oral appliance for providing sleep apnea relief comprising:
a body, the body formed in a u-shape ending in a rear portion having, a first end and a second end;
a first cross bracket connecting the first and second ends;
a second cross bracket connecting the first and second ends, the first and second cross brackets adapted to operate as structural elements to support a tongue depressor; and
a tongue depressor for connecting with at least one of the first and second cross brackets.

2. The oral appliance as set forth in claim 1, wherein body includes two base connectors and the tongue depressor includes two tongue connectors for selectively connecting with the corresponding base connector.

3. The oral appliance as set forth in claim 2, wherein the tongue depressor includes a tongue catch for connecting with the first cross bracket.

4. The oral appliance as set forth in claim 3, wherein the tongue depressor includes a rear tongue connector for connecting with the second cross bracket.

5. The oral appliance as set forth in claim 4, wherein when the tongue depressor connects with the body, it is adapted to be snapped into mating engagement with the body and pivoted about a snap axis.

6. The oral appliance as set forth in claim 1, wherein the body includes a base layer sandwiched between a top layer and a bottom layer, and wherein pliable members are formed on an exterior surface of at least one of the top layer and bottom layer.

7. An oral appliance for providing sleep apnea relief, comprising:
a body, the body formed in a u-shape ending in a rear portion having a first end and a second end;
a malleable spine molded into the body and protruding from at least one of the first and second ends; and
a toilette depressor connected with the malleable spine, thereby allowing a user to bend the malleable spine to position the tongue depressor into the desired position.

8. The oral appliance as set forth in claim 7, wherein the tongue depressor has a hole therethrough to allow saliva to pass through the tongue depressor.

9. The oral appliance as set forth in claim 8, wherein the body includes a base layer sandwiched between a top layer and a bottom layer, wherein the top and bottom layers are formed of a heat formable plastic that is adapted to molded upon exposure to boiling water.

10. The oral appliance as set forth in claim 9, wherein the spine is a malleable spine formed of a metal.

11. The oral appliance as set forth in claim 10, wherein the spine includes a retention portion and the tongue depressor is rotatably connected with the retention portion, and wherein the spine includes a plurality of protrusions of differing lengths, whereby a user can selectively adjust the tongue depressor by rotating the tongue depressor until the desired length is selected at a position to engage with a tongue when in use.

12. The oral appliance as set forth in claim 11, wherein the spine is connected to and between the first and second ends of the rear portion.

13. The oral appliance as set forth in claim 10 wherein the spine protrudes separately from each of the first and second ends of the rear portion such that the spine includes two separate retention portions, each with its own tongue depressor connected thereto.

14. The oral appliance as set forth in claim 13, wherein each of the tongue depressors is adapted to be bent and extended independently to push on a user's tongue at different points.

15. The oral appliance as set forth in claim 7, wherein the tongue depressor is formed to include a funneling trough to allow for the drainage of saliva.

16. The oral appliance as set forth in claim 15, wherein the spine is spring biased to force the tongue depressor down and against a user's tongue.

17. The oral appliance as set forth in claim 16, wherein an articulation point exists between the tongue depressor and body, such that both a cross-section of the body and tongue depressor at the articulation point is circular to provide a smooth exterior surface at all angular settings.

18. The oral appliance as set forth in claim 7, wherein the body includes a base layer sandwiched between a top layer and a bottom layer, and wherein pliable members are formed on an exterior surface of at least one of the top layer and bottom layer.

19. An oral appliance for providing sleep apnea relief, comprising:
a body, the body formed in a u-shape ending in a rear portion having a first end and a second end;
a tongue depressor rotatably connected with the body proximate each of the first and second ends, wherein an articulation point exists between the tongue depressor and body, such that both a cross-section of the body and tongue depressor at the articulation point is circular to provide a smooth exterior surface at all angular settings.

20. An oral appliance for providing sleep apnea relief, comprising:
a body, the body formed in a u-shape ending in a rear portion having a first end and a second end, wherein each of the first and second ends include a connector; and
a tongue depressor, the tongue depressor including connectors for selectively connecting with the connectors on the first and second ends, whereby a user can selectively select a tongue depressor with the desired dimensions and attached the selected tongue depressor with the body.
